(19) United States
(12) Patent Application Publication
(10) Pub. No.: US 2026/0268508 A1
(43) Pub. Date: Sep. 10, 2026

(54) SYSTEM AND METHOD FOR REGISTRATION OF THREE-DIMENSIONAL OCT/A IMAGES TO TRACK RETINAL AND CHOROIDAL STRUCTURAL CHANGES

(71) Applicant: UNIVERSITY OF PITTSBURGH - OF THE COMMONWEALTH SYSTEM OF HIGHER EDUCATION, PITTSBURGH, PA (US)

(72) Inventors: Shaohua Pi, Wexford, PA (US); Bingjie Wang, Wexford, PA (US)

(73) Assignee: UNIVERSITY OF PITTSBURGH - OF THE COMMONWEALTH SYSTEM OF HIGHER EDUCATION, PITTSBURGH, PA (US)

(21) Appl. No.: 19/160,889

(22) PCT Filed: Mar. 26, 2024
(86) PCT No.: PCT/US2024/021427

§ 371 Date: Aug. 29, 2025

Related U.S. Application Data
(60) Provisional application No. 63/492,527, filed on Mar. 28, 2023.

(51) Int. Cl. G06T 7/32; A61B 3/00; A61B 3/10; G06T 7/00

(52) U.S. Cl. CPC G06T 7/32; A61B 3/0025; A61B 3/102; G06T 7/0016; G06T 2200/04; G06T 2207/10101; G06T 2207/30041

(57) ABSTRACT

A system and method for registering OCT/A volumes includes receiving a first OCT/A volume and a second OCT/A volume, generating a first en face angiogram image for the first OCT/A volume and a second en face angiogram image for the second OCT/A volume, determining a lateral transformation coefficient matrix based on the first en face angiogram image and the second en face angiogram image, applying the lateral transformation coefficient matrix to the second OCT/A volume to create a laterally registered second OCT/A volume, determining a depth transformation matrix (DTM) based on the first OCT/A volume and the laterally registered second OCT/A volume, and applying the DTM to the laterally registered second OCT/A volume to create a laterally and axially registered second OCT/A volume. The systema and method may further include creating a 3-D change map based on the first OCT/A volume and the laterally and axially registered second OCT/A volume.

### CROSS REFERENCE TO RELATED APPLICATIONS

This application claims priority to U.S. Provisional Patent Application Ser. No. 63/492,527, filed on Mar. 28, 2023 and titled “System and Method for Registration of Three-Dimensional OCT/A Images to Track Retinal and Choroidal Structural Changes Volumetrically,” the disclosure of which is incorporated herein by reference.

### FIELD OF THE INVENTION

The disclosed concept relates generally to the diagnosis and treatment of retinal diseases, such as central serous chorioretinopathy (CSCR), and, in particular, to a system and method for registering 3-D OCT/A images acquired at different times and/or in different visits to a caregiver to track retinal and choroidal structural changes volumetrically.

### BACKGROUND OF THE INVENTION

Central serous chorioretinopathy (CSCR) is a vision-threatening condition caused by the breakdown of the barrier function of retinal pigment epithelium (RPE). The hallmark of CSCR is the accumulation of fluid within the sensory retina and RPE, leading to serous retinal detachment. Currently, observation is the standard of care for newly presenting CSCR. In most cases, acute CSCR is typically self-limiting, with retinal pigment epithelial detachment (PED) resolution and subretinal fluid (SRF) reabsorption spontaneously within three months. However, up to 50% of acute cases can recur within one year, and 15% of acute cases may have persistent SRF longer than six months. These cases can result in chronic CSCR that can cause permanent damage to photoreceptors and may require treatment. Since there is no consensus on the best therapies for CSCR, long-term follow-up visits are critical to monitor disease progression and determine the appropriate course of action.

Optical coherence tomography (OCT) has enabled the acquisition of high-resolution, three-dimensional (3D) retinal and choroidal images, and has been a routine imaging modality in the clinic. OCT has greatly improved the management of CSCR by allowing for the visualization of delayed resolution of SRF and abnormalities of RPE that are associated with a high risk of CSCR recurrence. Furthermore, measurements of choroidal thickness obtained using OCT have been used to evaluate the progression of CSCR. Additionally, OCT angiography (OCT/A), which allows for the visualization of retinal and choroidal circulations, has been used to identify specific vascular impairments associated with CSCR. However, these OCT/A evaluations are primarily performed in 2-D cross-sectional or en face images, which may not fully capture the 3-D nature of the retina and pathologies. Recently, 3-D quantification algorithms of OCT/A scans are emerging for better characterization of retinopathies in various diseases. In a previous study (B. Wang, A. Camino, S. Pi, Y. Guo, J. Wang, D. Huang, T. S. Hwang, and Y. Jia, “Three-dimensional structural and angiographic evaluation of foveal ischemia in diabetic retinopathy: method and validation,” Biomedical Optics Express 10, 3522-3532 (2019)), one of the present inventors developed a 3-D para-fovea vessel density (3D-PFVD) algorithm that achieved improved identification of foveal ischemia in diabetic retinopathy. Based on these findings, it was hypothesized that a 3-D quantification of the structural changes in CSCR might provide a better evaluation of the disease process and progression.

However, volumetric comparison of OCT/A scans acquired at different visits can be challenging due to the variation of pupil entrance for the scanning light beam (retinal curvature mismatch), shift of scanning regions (field mismatch), variation of transparency (reflectance mismatch), and eye movements (texture mismatch). All of these factors can introduce inaccuracies in biomarker detection and measurement. To align two OCT/A volumes for comparison, registration is necessary. While the prior art includes a volumetric registration algorithm for merging OCT/A scans from the same image session in rats (S. Pi, T. T. Hormel, X. Wei, W. Cepurna, J. C. Morrison, and Y. Jia, “Imaging retinal structures at cellular-level resolution by visible-light optical coherence tomography,” Optics letters 45, 2107-2110 (2020)), aligning and comparing OCT/A scans in humans and from different visits can be difficult due to substantial retinal structural deformation, especially for depth profiles in patients.

### SUMMARY OF THE INVENTION

In one embodiment, a method for registering a pair of OCT/A volumes is provided. The method includes receiving a first OCT/A volume and a second OCT/A volume for a patient, generating a first en face angiogram image for the first OCT/A volume and a second en face angiogram image for the second OCT/A volume, determining a lateral transformation coefficient matrix based on the first en face angiogram image and the second en face angiogram image, applying the lateral transformation coefficient matrix to the second OCT/A volume to create a laterally registered second OCT/A volume, determining a depth transformation matrix (DTM) based on the first OCT/A volume and the laterally registered second OCT/A volume, and applying the DTM to the laterally registered second OCT/A volume to create a laterally and axially registered second OCT/A volume. The method may further include creating a 3-D change map based on the first OCT/A volume and the laterally and axially registered second OCT/A volume.

In another embodiment, a system for registering a pair of OCT/A volumes is provided. The system includes a processing apparatus structured and configured to receive a first OCT/A volume and a second OCT/A volume for a patient, generate a first en face angiogram image for the first OCT/A volume and a second en face angiogram image for the second OCT/A volume, determine a lateral transformation coefficient matrix based on the first en face angiogram image and the second en face angiogram image, apply the lateral transformation coefficient matrix to the second OCT/A volume to create a laterally registered second OCT/A volume, determine a depth transformation matrix (DTM) based on the first OCT/A volume and the laterally registered second OCT/A volume, and apply the DTM to the laterally registered second OCT/A volume to create a laterally and axially registered second OCT/A volume.

### BRIEF DESCRIPTION OF THE DRAWINGS

A full understanding of the invention can be gained from the following description of the preferred embodiments when read in conjunction with the accompanying drawings in which:

FIG. 1 is flowchart illustrating an improved automated volumetric registration method for aligning OCT/A scans from baseline and follow-up visits in patients with CSCR according to an exemplary embodiment of the disclosed concept

FIGS. 2A-2H together comprise a schematic diagram illustrating the registration method of FIG. 1 according to an exemplary embodiment;

FIG. 3 is flowchart illustrating a method of accounting and compensating for variations in signal strength index that may affect OCT reflectance values according to an exemplary embodiment of the disclosed concept;

FIGS. 4A-4E together comprise a schematic diagram illustrating the compensation method of FIG. 3 according to an exemplary embodiment; and

FIG. 5 is a schematic diagram of an exemplary system for registering 3-D OCT/A images according to an exemplary embodiment of the disclosed concept.

### DETAILED DESCRIPTION OF THE INVENTION

As used herein, the singular form of “a”, “an”, and “the” include plural references unless the context clearly dictates otherwise.

As used herein, the statement that two or more parts or components are “coupled” shall mean that the parts are joined or operate together either directly or indirectly, i.e., through one or more intermediate parts or components, so long as a link occurs.

As used herein, “directly coupled” means that two elements are directly in contact with each other.

As used herein, the term “number” shall mean one or an integer greater than one (i.e., a plurality).

As used herein, the terms “component” and “system” are intended to refer to a computer related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

The disclosed concept will now be described, for purposes of explanation, in connection with numerous specific details in order to provide a thorough understanding of the disclosed concept. It will be evident, however, that the disclosed concept can be practiced without these specific details without departing from the spirit and scope of this innovation.

As described herein, the disclosed concept provides a methodology for registering OCT scans acquired from different visits without requiring prior layer segmentation and for calculating and creating three-dimensional (3-D) structural change maps for patients with CSCR. The disclosed concept, in its various exemplary embodiments, provides an improved automated volumetric registration algorithm that can align and compare OCT/A scans from baseline and follow up visits. Using this algorithm, 3-D structural change maps between the scans are generated to reveal the progression and resolution of pathologies in CSCR patients. The methodology can detect subtle retinal and choroidal changes and therefore can be a valuable tool for monitoring disease progression, guiding treatment decisions, and monitoring the effectiveness of any treatment given to CSCR patients.

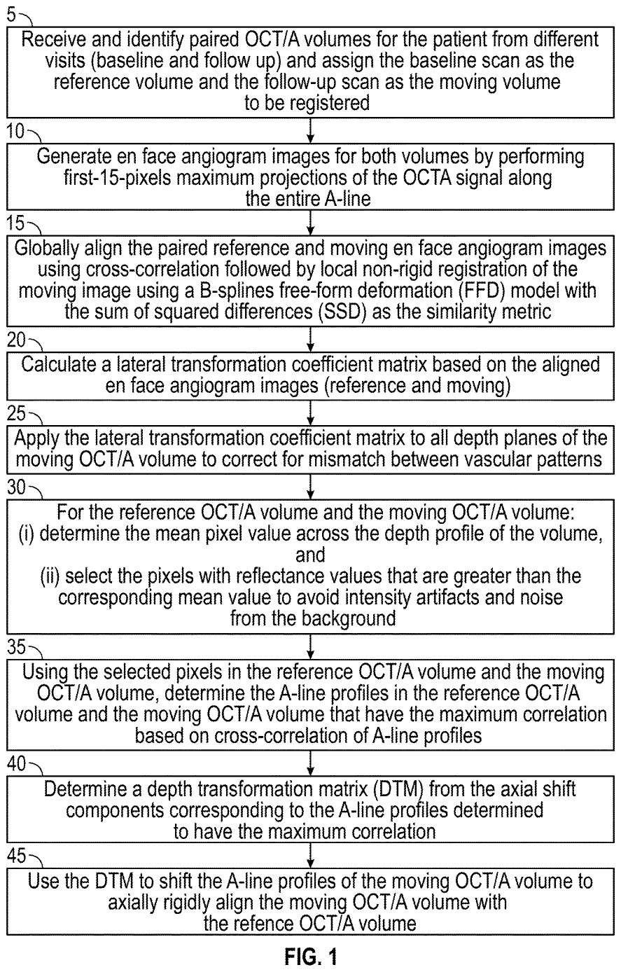

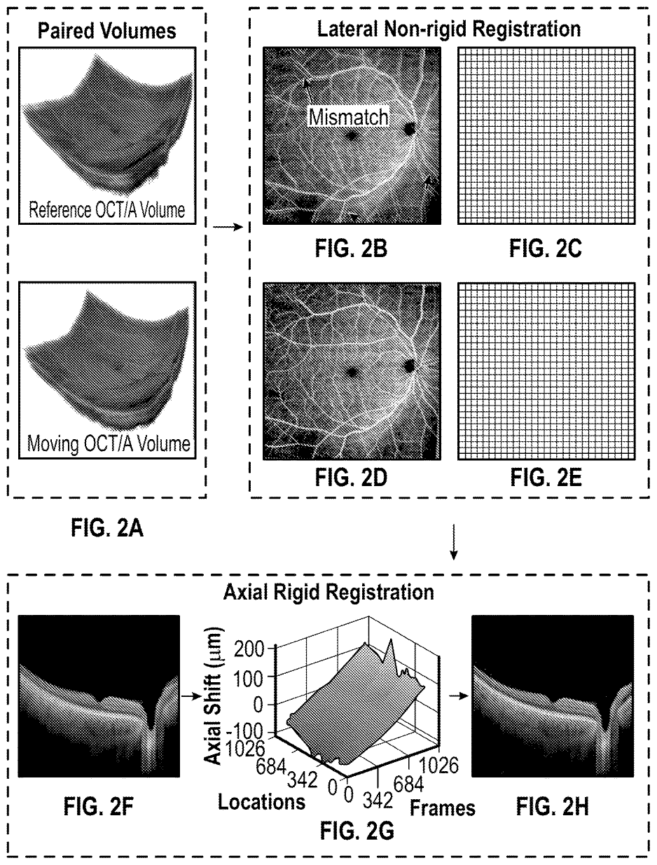

FIG. 1 is flowchart illustrating an improved automated volumetric registration method for aligning OCT/A scans from baseline and follow-up visits in patients with CSCR according to an exemplary embodiment of the disclosed concept. FIGS. 2A-2H together comprise a schematic diagram illustrating the registration method of FIG. 1 according to an exemplary embodiment, wherein FIG. 2A shows exemplary paired OCT/A volumes from different visits, FIGS. 2B-2E show the lateral non-rigid registration of the paired volumes (including en face angiogram images projected from the entire depth of the volumes for overlay before registration (FIG. 2B) and for overlay after registration (FIG. 2C)), and FIGS. 2F-2H show axial rigid registration, including overlay of cross-sectional images before axial registration (FIG. 2F) and after axial registration (FIG. 2H)).

Referring to FIG. 1, the method begins at step 5, wherein paired OCT/A volumes for the patient from different visits (baseline and follow up) are received. The received baseline scan is identified and assigned as the reference volume and the received follow-up scan is identified as the moving volume to be registered. This is shown in FIG. 2A. In the exemplary embodiment, OCT/A volumes are centered at the fovea, covering a 12 mm×12 mm field-of-view, and are obtained to capture the majority of the retina, including the fovea, optic disc, and peripheral regions. The known complex optical microangiography (OMAG) algorithm may be used to generate the OCT/A volumes. To minimize potential motion-artifacts during imaging, FastTrac motion correction (Carl Zeiss Meditec Inc., Dublin, CA), which detects and tracks eye motion via line-scanning ophthalmoscope (LSO) fundus imaging, may be employed. Next, at step 10, an en face angiogram image is generated for each volume by performing first-15-pixels maximum projections of the OCTA signal for the volume along the entire A-line. Then, at step 15, the paired reference and moving en face angiogram images are globally aligned using cross-correlation followed by local non-rigid registration of the moving image using a B-splines free-form deformation (FFD) model with the sum of squared differences (SSD) as the similarity metric. Next, at step 20, a lateral transformation coefficient matrix is calculated based on the aligned en face angiogram images (reference and moving), and at step 25, the lateral transformation coefficient matrix is applied to all depth planes of the moving OCT/A volume to correct for mismatch between vascular patterns. These steps are shown in FIGS. 2B-2E. At this point in the method, the lateral registration portion has been completed and a laterally corrected moving OCT/A volume has been created, i.e., the moving OCT/A volume has been laterally registered with the reference OCT/A volume.

The method then proceeds to step 30 for the beginning of the axial registration portion of the method. At step 30, for the reference OCT/A volume and the laterally aligned moving OCT/A volume: (i) the mean pixel value across the depth profile of the volume is determined, and (ii) the pixels with reflectance values that are greater than the corresponding mean value are selected to avoid intensity artifacts and noise from the background. Then, at step 35, using the selected pixels in the reference OCT/A volume and the laterally aligned moving OCT/A volume, the A-line profiles in the reference OCT/A volume and the laterally aligned moving OCT/A volume that have the maximum correlation based on cross-correlation of A-line profiles are determined. Next, at step 40, a depth transformation matrix (DTM) is determined from the axial shift components that correspond to the A-line profiles determined in step 35 to have the maximum correlation. Since the swellings caused by SRF and PED in eyes with CSCR may change between visits, the corresponding A-line profiles may be dramatically deformed in those regions, making the generated DTM not follow the trend within the field of view and causing local irregular values. Thus, in one particular (optional) embodiment, a Gaussian filter may be applied to the DTM and subtracted from the original DTM to localize those regions and then fill the regions using inward interpolation to create an adjusted DTM. Finally, at step 45, the DTM (original or adjusted) is used to shift the A-line profiles of the laterally aligned moving OCT/A volume to axially and rigidly align the moving OCT/A volume with the reference OCT/A volume. At this point, the moving OCT/A volume will be both laterally and axially registered with the reference OCT/A volume.

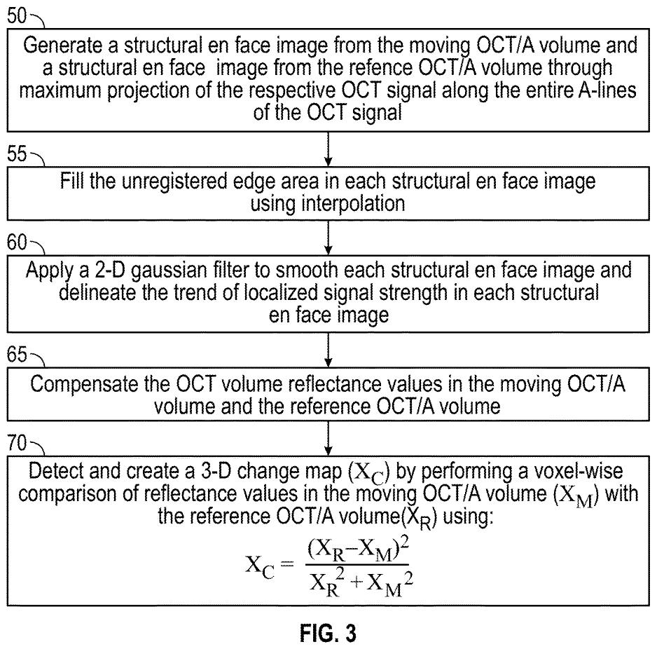

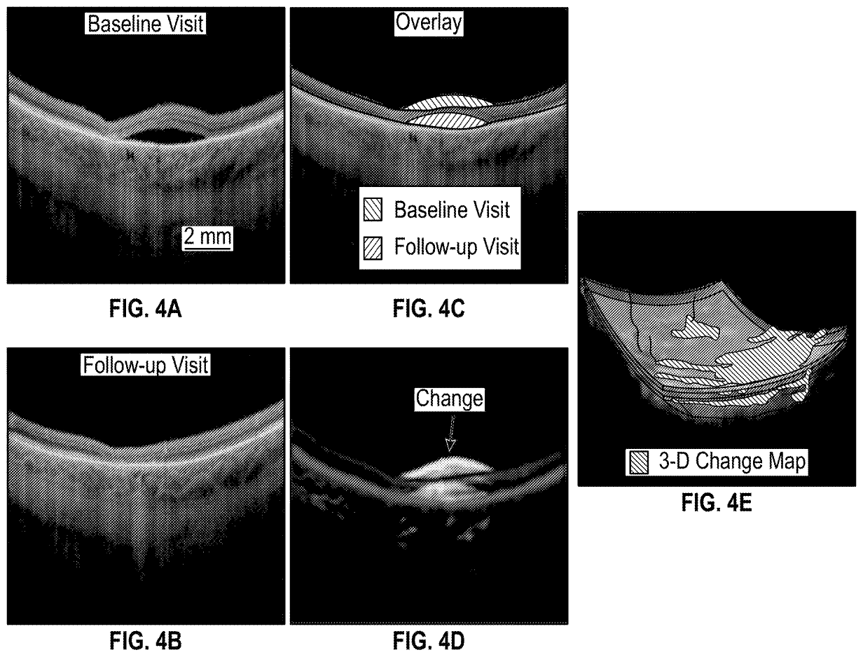

FIG. 3 is flowchart illustrating a method of accounting for and compensating for variations in signal strength index (SSI) that may affect OCT reflectance values to ensure a reliable volumetric comparison after registration as described above, including the generation of a 3-D change map, according to an exemplary embodiment of the disclosed concept. FIGS. 4A-4E comprise a schematic diagram illustrating the method of FIG. 3 according to an exemplary embodiment, wherein FIGS. 4A-4C show representative B-scans of a baseline visit with accumulated fluid in the subretinal space (FIG. 4A), a follow-up visit where the fluid is resolved (FIG. 4B), and overlay after registration (FIG. 4C), and wherein FIGS. 4D and 4E show a 3-D change map overlaid with a structural B-scan image (FIG. 4D) and volume (FIG. 4E).

Referring to FIG. 3, the method begins at step 50, wherein a structural en face image from the moving OCT/A volume is generated and a structural en face image from the reference OCT/A volume through maximum projection of the respective OCT signal along the entire A-lines of the OCT signal. Then, at step 55, the unregistered edge area in each structural en face image is filled using interpolation. Next, at step 60, a 2-D Gaussian filter is applied to smooth each structural en face image to delineate the trend of localized signal strength in each structural en face image. Thereafter, at step 65, the OCT volume reflectance values in the moving OCT/A volume and the reference OCT/A volume are compensated by dividing each depth plane of the moving OCT/A volume by the smoothed structural en face image from moving OCT/A and dividing each depth plane of the reference OCT/A volume by the smoothed structural en face image from reference OCT/A. Then, at step 70, a 3-D change map (X_{C}) is detected and created by performing a voxel-wise comparison of reflectance values in the moving OCT/A volume (X_{M}) with the reference OCT/A volume (X_{R}) using the following formula:

$${X}_{C} = \frac{{( {X}_{R} - {X}_{M} )}^{2}}{{X}_{R}^{2} + {X}_{M}^{2}} .$$

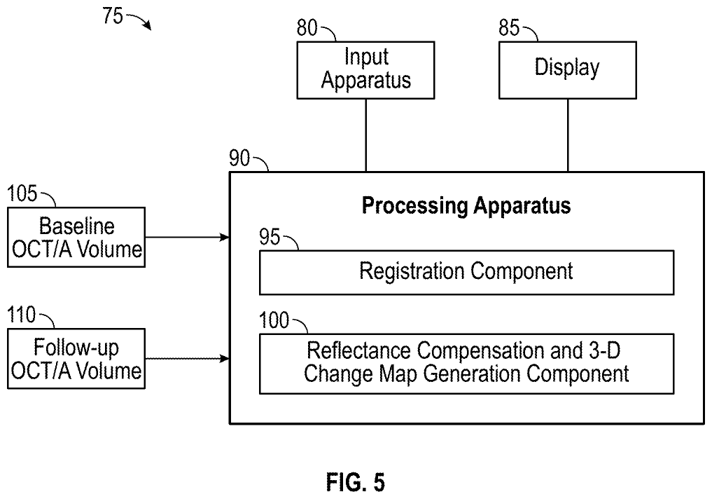

FIG. 5 is a schematic diagram of an exemplary system 75 for registering 3-D OCT/A images according to an exemplary embodiment of the disclosed concept. As seen in FIG. 5, system 75 is a computing device structured and configured to receive a baseline OCT/A volume 105 and a follow-up OCT/A volume 110 and register those volumes and generate a 3-D change map using those volumes as described herein. System 75 may comprise, for example and without limitation, a PC, a laptop computer, a tablet computer, or any other suitable computing device structured and configured to perform the functionality described herein. System 75 includes an input apparatus 80 (such as a keyboard), a display 85 (such as an LCD), and a processing apparatus 90. A user, such as a caregiver, is able to provide input into processing apparatus 90 using input apparatus 80, and processing apparatus 90 provides output signals to display 85 to enable display 85 to display information to the user for tracking retinal and choroidal structural changes volumetrically as described herein. Processing apparatus 90 comprises a processor and a memory. The processor may be, for example and without limitation, a microprocessor (μP), a microcontroller, an application specific integrated circuit (ASIC), or some other suitable processing device that interfaces with the memory. The memory can be any one or more of a variety of types of internal and/or external storage media such as, without limitation, RAM, ROM, EPROM(s), EEPROM(s), FLASH, and the like that provide a storage register, i.e., a non-transitory machine readable medium, for data storage such as in the fashion of an internal storage area of a computer, and can be volatile memory or nonvolatile memory. The memory has stored therein a number of routines that are executable by the processor, including routines for implementing one or more of the exemplary embodiments of the disclosed concept as described herein.

In particular, processing apparatus 90 includes a registration component 95 configured for laterally and axially registering baseline OCT/A volume 105 and follow-up OCT/A volume 110. In the non-limiting exemplary embodiment, registration component 95 implements the exemplary method shown in FIG. 1. Processing apparatus 90 further includes a reflectance compensation and 3-D change map generation component 100 configured for accounting for and compensating for variations in signal strength index (SSI) that may affect OCT reflectance values in baseline OCT/A volume 105 and follow-up OCT/A volume 110 and for generating a 3-D change map from baseline OCT/A volume 105 and follow-up OCT/A volume 110. In the non-limiting exemplary embodiment, reflectance compensation and 3-D change map generation component 100 implements the exemplary method shown in FIG. 3.

Thus, as described herein, the disclosed concept provides an improved automated volumetric registration algorithm to align OCT/A scans from baseline and follow-up visits in patients with CSCR. The disclosed concept compensates signal strength variation and can align OCT/A scans regardless of the structural changes. After registration, 3-D structural change maps can be obtained to evaluate the progression and resolution of pathologies in CSCR patients by performing volumetric comparison of the scans. The algorithm of the disclosed concept has been shown to achieve great efficiency with high Jaccard coefficients of large vessel masks laterally and negligible centers of mass of A-lines axially.

The algorithm of the disclosed concept has several advantages over traditional method of volumetric registration. First, it eliminates the need for prior layer segmentation, which can be a time-consuming and error-prone process. Second, by aligning the entire volume, rather than just specific layers, this algorithm allows for a more comprehensive analysis of the entire retina and choroid with a more accurate overall picture of the disease. It can detect changes that may be invisible in a layer-based analysis. Third, although developed and demonstrated in CSCR, the algorithm may also be extremely helpful for screening to monitor changes in the retina and choroid over time in healthy subjects during normal aging to detect early signs of retinal damage. Additionally, the algorithm may also be applied in other retinal diseases with more complex structural and vascular changes.

In addition, the present inventors have observed that the 2-D en face projected structural changes of the retina and choroid were not similar with low Jaccard coefficients, indicating the algorithm of the disclosed concept was able to capture different patterns of structural change of the retina and choroid over time and calculate them separately. This finding may have important implications for understanding the underlying mechanisms of CSCR progression or conditions as well as for developing targeted and more effective treatments. Moreover, the present inventors compared the sum of structural change volume in SRF, PED and retinal thickness, and the change volumes in the retina calculated from 3-D change maps. The results indicated that the 3-D change maps can reliably detect structural changes of SRF, PED, and thickness caused by CSCR simultaneously.

In short, the disclosed concept presents an improved automated volumetric registration algorithm to align OCT scans for comparison. The algorithm eliminates the need for prior layer segmentation and enables the detection of volumetric changes in the entire OCT scan region efficiently. The accurate and sensitive identification of the pathology resolution after treatment, as well as tracking recurrence and progression, can be useful in predicting treatment response and evaluating treatment efficacy in patients with CSCR. It provides valuable insights into the potential use of OCT/A in the diagnosis and management of CSCR and highlights the need for continued research in this area. The algorithm of the disclosed concept may be integrated into clinical practice to provide a more accurate and efficient way of monitoring the progression of retinal diseases and assessing treatment efficacy in the near future.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of disclosed concept which is to be given the full breadth of the claims appended and any and all equivalents thereof.

What is claimed is:

1. A method for registering a pair of OCT/A volumes, comprising:
receiving a first OCT/A volume and a second OCT/A volume for a patient;
generating a first en/ice angiogram image for the first OCT/A volume and a second en face angiogram image for the second OCT/A volume;
determining a lateral transformation coefficient matrix based on the first en face angiogram image and the second en/ace angiogram image;
applying the lateral transformation coefficient matrix to the second OCT/A volume to create a laterally registered second OCT/A volume;
determining a depth transformation matrix (DTM) based on the first OCT/A volume and the laterally registered second OCT/A volume; and
applying the DTM to the laterally registered second OCT/A volume to create a laterally and axially registered second OCT/A volume.

2. The method according to claim 1, wherein the determining the lateral transformation coefficient matrix includes: (i) globally aligning the first en fee angiogram image and the second en face angiogram image using cross-correlation, (ii) performing local non-rigid registration of the second en face angiogram image with respect to the first en face angiogram image; and (iii) calculating the lateral transformation coefficient matrix based on the non-rigidly registered second enface angiogram image.

3. The method according to claim 1, wherein the lateral transformation coefficient matrix is applied to all depth planes of the second OCT/A volume to create the laterally registered second OCT/A volume.

4. The method according to claim 1, wherein the determining the DTM is based on a cross correlation of the first OCT/A volume and the laterally registered second OCT/A volume.

5. The method according to claim 4, further comprising calculating the cross-correlation based on A-line profiles of the first OCT/A volume and the laterally registered second OCT/A volume.

6. The method according to claim 5, wherein the determining the DTM is based on axial shift components corresponding to the A-line profiles of the first OCT/A volume and the laterally registered second OCT/A volume that are determined to have a maximum correlation.

7. The method according to claim 2, wherein the performing the local non-rigid registration of the second en face angiogram images uses a B-splines free-form deformation (FFD) model with the sum of squared differences (SSD) as a similarity metric.

8. The method according to claim 1, wherein the DTM is applied to A-line profiles of the laterally registered second OCT/A volume to create the laterally and axially registered second OCT/A volume.

9. The method according to claim 1, wherein the DTM is created by creating an original DTM based on the first OCT/A volume and the laterally registered second OCT/A volume and applying a Gaussian filter to the original DTM to localize particular regions and filling the particular regions using inward interpolation to create the DTM.

10. The method according to claim 1, further comprising creating a 3-D change map based on the first OCT/A volume and the laterally and axially registered second OCT/A volume.

11. The method according to claim 1, wherein the 3-D change map (X_{C}) is created by performing a voxel-wise comparison of first reflectance values obtained from the first OCT/A volume (X_{R}) and second reflectance values obtained from the laterally and axially registered second OCT/A volume (X_{M}) using the following formula:
$${X}_{C} = \frac{{( {X}_{R} - {X}_{M} )}^{2}}{{X}_{R}^{2} + {X}_{M}^{2}} .$$

12. The method according to claim 11, wherein the first reflectance values include compensation for variations in signal strength index (SSI) based on a structural en face image obtained from the first OCT/A volume and wherein the reflectance values include compensation for variations in signal strength index (SSI) based on a structural en face image obtained from the laterally and axially registered second OCT/A volume.

13. The method according to claim 1, wherein the first OCT/A volume and a second OCT/A volume are obtained for an eye of the patient centered at a fovea of the eye.

14. A computer program product, comprising a non-transitory computer usable medium having a computer readable program code embodied therein, the computer readable program code being adapted to be executed to implement a method for registering a pair of OCT/A volumes as recited in claim 1.

15. A system for registering a pair of OCT/A volumes, comprising:
a processing apparatus structured and configured to:
receive a first OCT/A volume and a second OCT/A volume for a patient;
generate a first en face angiogram image for the first OCT/A volume and a second enface angiogram image for the second OCT/A volume;
determine a lateral transformation coefficient matrix based on the first en face angiogram image and the second en face angiogram image;
apply the lateral transformation coefficient matrix to the second OCT/A volume to create a laterally registered second OCT/A volume;
determine a depth transformation matrix (DTM) based on the first OCT/A volume and the laterally registered second OCT/A volume; and
apply the DTM to the laterally registered second OCT/A volume to create a laterally and axially registered second OCT/A volume.

16. The system according to claim 15, wherein the processing apparatus is structured and configured to determine the lateral transformation coefficient matrix by: (i) globally aligning the first en face angiogram image and the second enface angiogram image using cross-correlation, (ii) performing local non-rigid registration of the second en face angiogram image with respect to the first en face angiogram image; and (iii) calculating the lateral transformation coefficient matrix based on the non-rigidly registered second en face angiogram image.

17. The system according to claim 16, wherein the lateral transformation coefficient matrix is applied to all depth planes of the second OCT/A volume to create the laterally registered second OCT/A volume.

18. The system according to claim 16, wherein the processing apparatus is structured and configured to determine the DTM based on a cross correlation of the first OCT/A volume and the laterally registered second OCT/A volume.

19. The system according to claim 18, wherein the processing apparatus is structured and configured to calculate the cross-correlation based on A-line profiles of the first OCT/A volume and the laterally registered second OCT/A volume.

20. The system according to claim 19, wherein the processing apparatus is structured and configured to determine the DTM based on axial shift components corresponding to the A-line profiles of the first OCT/A volume and the laterally registered second OCT/A volume that are determined to have a maximum correlation.

21. The system according to claim 17, wherein the processing apparatus is structured and configured to perform the local non-rigid registration of the second en face angiogram images using a B-splines free-form deformation (FFD) model with the sum of squared differences (SSD) as a similarity metric.

22. The system according to claim 16, wherein the DTM is applied to A-line profiles of the laterally registered second OCT/A volume to create the laterally and axially registered second OCT/A volume.

23. The system according to claim 16, wherein the DTM is created by creating an original DTM based on the first OCT/A volume and the laterally registered second OCT/A volume and applying a Gaussian filter to the original DTM to localize particular regions and filling the particular regions using inward interpolation to create the DTM.

24. The system according to claim 16, wherein the processing apparatus is structured and configured to create a 3-D change map based on the first OCT/A volume and the laterally and axially registered second OCT/A volume.

25. The system according to claim 16, wherein the 3-D change map (X_{C}) is created by performing a voxel-wise comparison of first reflectance values obtained from the first OCT/A volume (X_{R}) and second reflectance values obtained from the laterally and axially registered second OCT/A volume (X_{M}) using the following formula:
$${X}_{C} = \frac{{( {X}_{R} - {X}_{M} )}^{2}}{{X}_{R}^{2} + {X}_{M}^{2}} .$$

26. The system according to claim 25, wherein the first reflectance values include compensation for variations in signal strength index (SSI) based on a structural en face image obtained from the first OCT/A volume and wherein the reflectance values include compensation for variations in signal strength index (SSI) based on a structural en face image obtained from the laterally and axially registered second OCT/A volume.

27. The system according to claim 16, wherein the first OCT/A volume and a second OCT/A volume are obtained for an eye of the patient centered at a fovea of the eye.
